(19) United States
(12) Patent Application Publication
(10) Pub. No.: US 2026/0094296 A1
(43) Pub. Date: Apr. 2, 2026

(54) IMAGING ORIENTATION PLANNING FOR EXTERNAL IMAGING DEVICES

(71) Applicant: INTUITIVE SURGICAL OPERATIONS, INC., Sunnyvale, CA (US)

(72) Inventors: Hui Zhang, Palo Alto, CA (US); Federico Barbagli, San Francisco, CA (US); Energy Cruse, II, Foster City, CA (US)

(21) Appl. No.: 19/113,738

(22) PCT Filed: Sep. 22, 2023
(86) PCT No.: PCT/US2023/074837

§ 371 Date: Mar. 20, 2025

Related U.S. Application Data
(60) Provisional application No. 63/409,310, filed on Sep. 23, 2022.

(51) Int. Cl. G06T 7/73; A61B 6/03; A61B 6/50; A61B 34/10; A61B 34/30; A61B 34/35; G16H 30/40

(52) U.S. Cl. CPC G06T 7/73; A61B 6/032; A61B 6/50; A61B 34/35; G16H 30/40; A61B 2034/107; A61B 2034/301; G06T 2207/10081; G06T 2207/10124; G06T 2207/30008; G06T 2207/30061; G06T 2207/30168; G06T 2207/30244

(57) ABSTRACT

Methods and systems for forming one or more projected two-dimensional images based on Computed Tomography (CT) data of a subject, a planned pose of a flexible elongated device, and one or more parameters of an external imaging device are disclosed. In some examples, the one or more projected two-dimensional images may be used to determine appropriate imaging orientations of an external imaging device for use during a medical procedure.

### CROSS-REFERENCED APPLICATIONS

This application claims priority to and benefit of U.S. Provisional Application No. 63/409,310 filed Sep. 23, 2022 and entitled “Imaging Orientation Planning for External Imaging Devices,” which is incorporated by reference herein in its entirety.

### FIELD

Disclosed examples relate to imaging orientation planning for external imaging devices and related systems.

### BACKGROUND

C-arm fluoroscopes are often used to take x-rays of a patient on a patient table. Depending on the procedure being performed, a medical practitioner may reorient the C-arm to view the interior tissues and/or organs of a subject, and in some instances a flexible elongated device positioned within an anatomical structure of a subject, from multiple different orientations during a medical procedure. This may be done to help the medical practitioner determine the relative positions and orientations of the different tissue and flexible elongated device during the medical procedure.

### SUMMARY

In some examples, a method includes obtaining computed tomography (CT) data for a subject, wherein the CT data includes a target tissue of the subject; obtaining a planned pose of a flexible elongated device within an anatomical structure of the subject; and based on the CT data, the planned pose of the flexible elongated device, and one or more parameters of an external imaging device, forming one or more projected two dimensional images.

In some examples, a non-transitory computer readable memory includes instructions that when executed by one or more processors performs the above method.

In some examples, a medical procedure planning apparatus includes a processor; and non-transitory computer readable memory storing computer-executable instructions that, when executed by the processor, cause the apparatus to: obtain computed tomography (CT) data for a subject, wherein the CT data includes a target tissue of the subject; obtain a planned pose of a flexible elongated device within an anatomical structure of the subject; and based on the CT data, the planned pose of the flexible elongated device, and one or more parameters of an external imaging device, determine one or more imaging orientations of the external imaging device for use during a medical procedure.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting examples when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic of an illustrative teleoperated medical system, in accordance with examples of the present disclosure.

FIG. 2A illustrates a medical instrument system, in accordance with examples of the present disclosure.

FIG. 2B illustrates a distal end portion of a medical instrument system, in accordance with examples of the present disclosure.

FIG. 3 is an illustration of an external imaging device being operated with a subject in place, in accordance with examples of the present disclosure.

FIG. 4A is a schematic representation of a detector and source of an external imaging device relative to an object in a first orientation in accordance with examples of the present disclosure.

FIG. 4B is a schematic representation of a detector and source of an external imaging device relative to the object of FIG. 4A in a second orientation in accordance with examples of the present disclosure.

FIG. 5 is a flow diagram of a method for determining imaging orientations for use during a medical procedure in accordance with examples of the present disclosure.

FIGS. 6A-6B depict one example for forming projected two dimensional images using previously obtained external imaging data in two different orientations in accordance with examples of the present disclosure.

FIGS. 7A-7C depict computational tomography (CT) data of a subject's torso as viewed from different orientations in accordance with examples of the present disclosure.

FIG. 8A depicts computational tomography (CT) data of a subject's torso with a planned pose of a flexible elongated device and the location of a target tissue included in the CT data in accordance with examples of the present disclosure.

FIG. 8B is a projected two-dimensional view of the CT data of FIG. 8A in accordance with examples of the present disclosure.

FIG. 9A is a projected two dimensional image including a flexible elongated device and target tissue as viewed from a first orientation and with a minimum bounding area surrounding the flexible elongated device.

FIG. 9B is a projected two dimensional image of the flexible elongated device and target tissue of FIG. 9A as viewed from a second orientation and with a minimum bounding area surrounding the flexible elongated device.

### DETAILED DESCRIPTION

C-arm fluoroscopes may be used to provide fluoroscopic imaging of a subject in various medical procedures including, for example, interventional pulmonary procedures. During such a procedure, a medical practitioner may desire to position a distal portion of a flexible elongated device, such as a catheter and/or endoscope, or other appropriate flexible elongated device in a desired pose to interact with a target tissue. Due to C-arm fluoroscopes typically presenting real time two-dimensional x-ray images of the subject, it may be difficult for a medical practitioner to determine a relative pose of the flexible elongated device and target tissue in three-dimensional space. Accordingly, the medical practitioner may move the C-arm fluoroscope or other external imaging device between multiple orientations relative to the subject during a procedure to ensure appropriate positioning and orientation of the flexible elongated device during the procedure. Due to the medical practitioner typically not knowing which imaging orientations will need to be viewed to determine the relative pose of the flexible elongated device and target tissue, the medical practitioner may view the subject at multiple imaging orientations. This may increase the overall x-ray dosage delivered to the subject during the procedure.

In view of the above, the Inventors have recognized a desire to reduce the x-ray dosages a subject is exposed to during a procedure. Thus, the Inventors have recognized the benefits associated with determining appropriate imaging orientations of an external imaging device for viewing a flexible elongated device and target tissue within a subject prior to performing a medical procedure. By finding appropriate viewing orientations prior to performing the medical procedure, a medical practitioner may not need to manually search for optimal imaging orientations in real time during a procedure which may result in decreased x-ray doses as compared to typical unplanned procedures.

In some examples, appropriate imaging orientations of an external imaging device for use during a medical procedure may be determined by forming projected two-dimensional images using previously taken computed tomography (CT) data of a subject's anatomy. These projected two-dimensional images may correspond to different orientations of an external imaging device relative to a subject. Accordingly, the relative positioning and orientations of various anatomical structures and/or planned pose of a flexible elongated device within the subject may be viewed and/or otherwise evaluated in these projected two-dimensional images prior to performing a procedure. Thus, the projected two-dimensional images may be used to determine appropriate imaging orientations of an external imaging device for use during a medical procedure. As elaborated in further detail below, this may either be done manually by a medical practitioner selecting orientations based on their viewing of the projected two dimensional images and/or appropriate imaging orientations may be determined based at least in part on an analysis of the plurality projected two-dimensional images.

The disclosed methods and systems may be used for any appropriate application. For example, predetermined imaging orientations may be used in medical procedures associated with any desirable anatomical structure of a subjection including, but not limited to, medical procedures for the lungs, colon, intestines, kidneys, kidney calices, brain, heart, the circulatory system including vasculature, and/or any other appropriate type of medical procedure. These medical procedures may also include the use of any appropriate external imaging device that may be moved between different orientations relative to the subject including, but not limited to, x-ray imaging devices such as x-ray fluoroscopes including C-arm fluoroscopes, and/or any other appropriate external imaging device capable of imaging the interior anatomy of a subject. Additionally, the disclosed methods and systems may facilitate medical procedures involving the use of endoscopes, catheters, laparoscopes, and/or any other appropriate flexible elongated device that may be used in a medical procedure where real time x-ray imaging may be used.

As used herein, an orientation of an external imaging device may refer to the orientation of the source and detector of the imaging device relative to a subject during a planned medical procedure. For example, an orientation of an external imaging device may refer to a C-arm angle when the imaging device is a C-arm fluoroscope, though other external imaging devices and types of orientations may be used as noted above.

In the following description, specific details are set forth describing some examples consistent with the present disclosure. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one example may be incorporated into other examples unless specifically described otherwise or if the one or more features would make an example non-functional.

In some instances well known methods, procedures, components, and circuits have not been described in detail so as not to unnecessarily obscure aspects of the examples.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

Turning to the figures, specific non-limiting examples are described in further detail. It should be understood that the various systems, components, features, and methods described relative to these examples may be used either individually and/or in any desired combination as the disclosure is not limited to only the specific examples described herein.

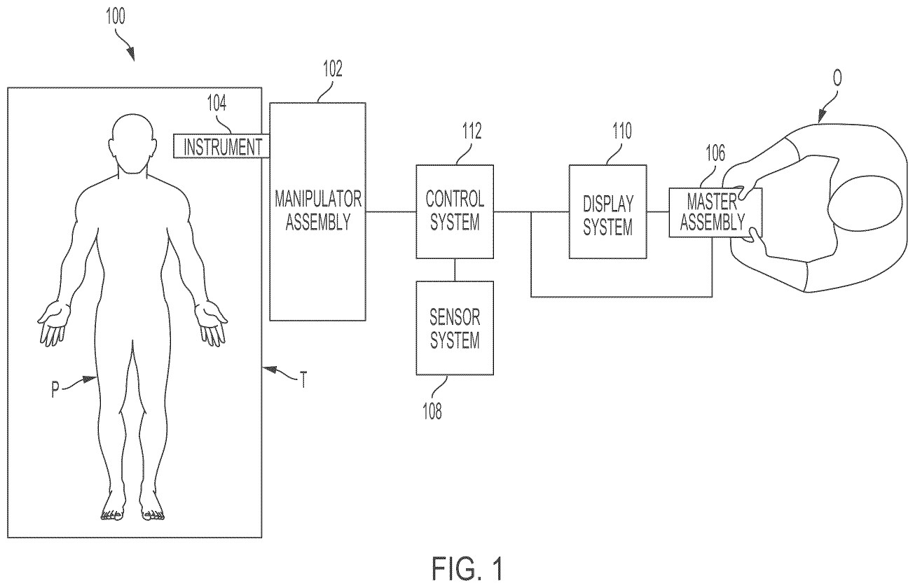

FIG. 1 is a simplified diagram of a teleoperated medical system 100 according to some examples. In some examples, teleoperated medical system 100 may be suitable for use in, for example, surgical, diagnostic, therapeutic, or biopsy procedures where it may be desirable to provide real time imaging of the medical procedure being performed. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

As shown in FIG. 1, medical system 100 generally includes a manipulator assembly 102 for operating a medical instrument 104 in performing various procedures on a patient P positioned on a table T. The manipulator assembly 102 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. Master assembly 106 generally includes one or more control devices for controlling manipulator assembly 102.

Manipulator assembly 102 supports medical instrument 104 and may optionally include a plurality of actuators or motors that drive inputs on medical instrument 104 in response to commands from a control system 112. The actuators may optionally include drive systems that when coupled to medical instrument 104 may advance medical instrument 104 into a naturally or surgically created anatomic orifice. Other drive systems may move the distal end of medical instrument 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 104 for grasping tissue in the jaws of a biopsy device and/or the like. Actuator position sensors such as resolvers, encoders, potentiometers, and other mechanisms may provide sensor data to medical system 100 describing the rotation and orientation of the motor shafts. This position sensor data may be used to determine motion of the objects manipulated by the actuators.

Teleoperated medical system 100 also includes a display system 110 for displaying an image or representation of the surgical site and medical instrument 104 generated by sub-systems of sensor system 108. Display system 110 and master assembly 106 may be oriented so operator O can control medical instrument 104 and master assembly 106 with the perception of telepresence.

In some examples, medical instrument 104 may include components of an imaging device, which may include an imaging scope assembly or imaging instrument that records a concurrent or real-time image of a medical site and provides the image to the operator or operator O through one or more displays of medical system 100, such as one or more displays of display system 110. The concurrent image may be, for example, a two or three-dimensional image captured by an imaging instrument positioned within the medical site. In some examples, the imaging device includes endoscopic imaging instrument components that may be integrally or removably coupled to medical instrument 104.

However, in some examples, a separate endoscope, attached to a separate manipulator assembly may be used with medical instrument 104 to image the medical site. In some examples, as described in detail below, the imaging instrument alone or in combination with other components of the medical instrument 104 may include one or more mechanisms for cleaning one or more lenses of the imaging instrument when the one or more lenses become partially and/or fully obscured by fluids and/or other materials encountered by the distal end of the imaging instrument. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Publication No. WO/2016/025465 filed Aug. 11, 2016 disclosing “Systems and Methods for Cleaning an Endoscopic Instrument”; U.S. patent application Ser. No. 15/508,923 filed Mar. 5, 2017 disclosing “Devices, Systems, and Methods Using Mating Catheter Tips and Tools”; and U.S. patent application Ser. No. 15/503,589 filed Feb. 13, 2017 disclosing “Systems and Methods for Cleaning an Endoscopic Instrument,” each of which is incorporated by reference herein in its entirety. The imaging device may be implemented as hardware, firmware, software or a combination thereof which interact with or are otherwise executed by one or more computer processors, which may include the processors of the control system 112.

Teleoperated medical system 100 may also include control system 112. Control system 112 includes at least one memory and at least one computer processor (not shown) for effecting control between medical instrument 104, master assembly 106, sensor system 108, and display system 110. Control system 112 also includes programmed instructions (e.g., a non-transitory fluoroscope-readable medium storing the instructions) to implement some or all of the methods described in accordance with aspects disclosed herein, including instructions for providing information to display system 110.

Control system 112 may optionally further include a virtual visualization system to provide navigation assistance to operator O when controlling medical instrument 104 during an image-guided surgical procedure. Virtual navigation using the virtual visualization system may be based upon reference to an acquired preoperative or intraoperative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube x-ray imaging, and/or the like.

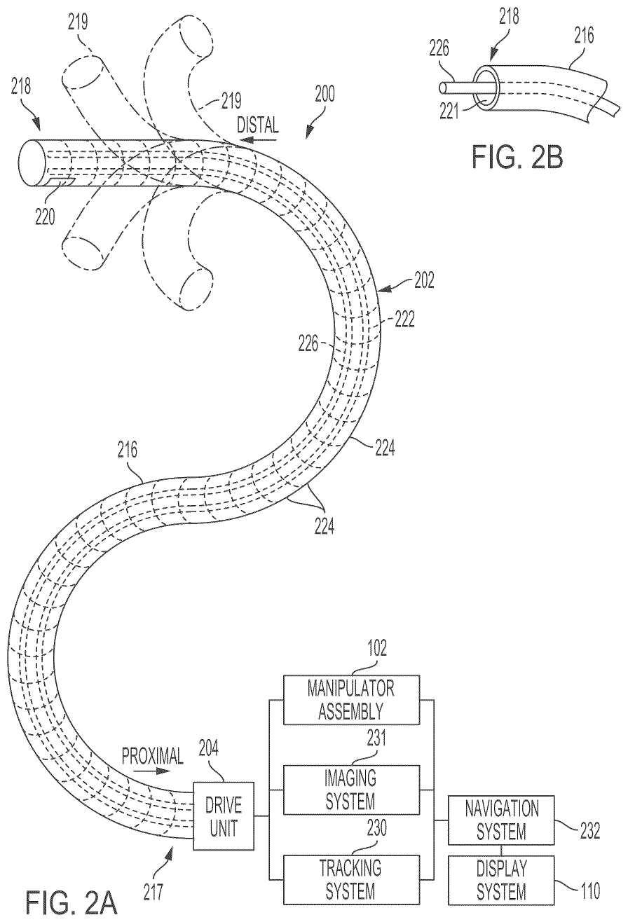

FIG. 2A is a simplified diagram of a medical instrument system 200 according to some examples. Medical instrument system 200 includes flexible elongated body 202, such as a flexible catheter, coupled to a drive unit 204. Flexible elongated body 202 includes a flexible elongated body 216 having proximal end 217 and distal end or tip portion 218. Medical instrument system 200 further includes a tracking system 230 for determining the position, orientation, speed, velocity, pose, and/or shape of distal end 218 and/or of one or more segments 224 along flexible elongated body 216 using one or more sensors and/or imaging devices as described in further detail below.

Tracking system 230 may optionally track distal end 218 and/or one or more of the segments 224 using a shape sensor 222. Shape sensor 222 may optionally include an optical fiber aligned with flexible elongated body 216 (e.g., provided within an interior channel (not shown) or mounted externally). The optical fiber of shape sensor 222 forms a fiber optic bend sensor for determining the shape of flexible elongated body 216. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the flexible elongated body may be determined using other techniques. For example, a history of the distal end pose of flexible elongated body 216 can be used to reconstruct the shape of flexible elongated body 216 over the interval of time. In some examples, tracking system 230 may optionally and/or additionally track distal end 218 using a position sensor system 220. Position sensor system 220 may be a component of an EM sensor system with position sensor system 220 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

Flexible elongated body 216 includes a channel 221 sized and shaped to receive a medical instrument 226. FIG. 2B is a simplified diagram of flexible elongated body 216 with medical instrument 226 extended according to some examples. In some examples, medical instrument 226 may be used for procedures such as surgery, biopsy, ablation, illumination, irrigation, or suction. Medical instrument 226 can be deployed through channel 221 of flexible elongated body 216 and used at a target location within the anatomy. Medical instrument 226 may include, for example, image capture probes, biopsy instruments, laser ablation fibers, and/or other surgical, diagnostic, or therapeutic tools. Medical instrument 226 may be used with an imaging instrument (e.g., an image capture probe) also within flexible elongated body 216. The imaging instrument may include a cable coupled to the camera for transmitting the captured image data. In some examples, the imaging instrument may be a fiber-optic bundle, such as a fiberscope, that couples to image processing system 231. The imaging instrument may be single or multi-spectral, for example capturing image data in one or more of the visible, infrared, and/or ultraviolet spectrums. Medical instrument 226 may be advanced from the opening of channel 221 to perform the procedure and then retracted back into the channel when the procedure is complete. Medical instrument 226 may be removed from proximal end 217 of flexible elongated body 216 or from another optional instrument port (not shown) along flexible elongated body 216.

Flexible elongated body 216 may also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch of distal end 218 and “left-right” steering to control a yaw of distal end 281. Steerable flexible elongated bodies are described in detail in U.S. patent application Ser. No. 13/274,208 (filed Oct. 14, 2011) (disclosing “Catheter with Removable Vision Probe”), which is incorporated by reference herein in its entirety.

The information from tracking system 230 may be sent to a navigation system 232 where it is combined with information from image processing system 231 and/or the preoperatively obtained models to provide the operator with real-time position information. In some examples, the real-time position information may be displayed on display system 110 of FIG. 1 for use in the control of medical instrument system 200. In some examples, control system 116 of FIG. 1 may utilize the position information as feedback for positioning medical instrument system 200. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” which is incorporated by reference herein in its entirety.

In some examples, medical instrument system 200 may be teleoperated within medical system 100 of FIG. 1. In some examples, manipulator assembly 102 of FIG. 1 may be replaced by direct operator control. In some examples, the direct operator control may include various handles and operator interfaces for hand-held operation of the instrument.

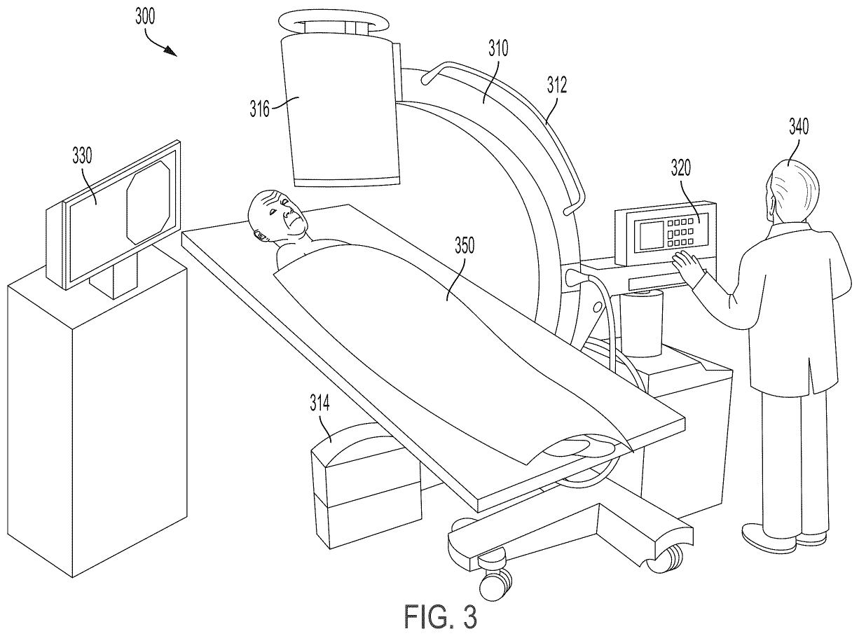

FIG. 3 is an illustration of an external imaging device being operated with a subject in place, in accordance with examples of the present disclosure. In some examples, such an external imaging device may be used in cooperation with the medical system 100 and/or medical instrument system 200 of FIGS. 1-2B to facilitate imaging of the flexible elongated device while it is positioned within the anatomy of a subject. FIG. 3 shows a manual C-arm imaging system as the external imaging device 300 in the depicted example.

However, it should be understood that an automatic C-arm or other external imaging device may be used as previously described. In the depicted example, the external imaging device 300 includes a C-arm 310, source 314, detector 316, and manual handle 312. In some examples, the external imaging device 300 includes a display 330. FIG. 3 also shows an operator 340 operating the manual handle 312 and a subject 350 being scanned by the external imaging device 300. The source 314 and detector 316 are rotatable around the subject as a pair such that a relative pose between the source 314 and detector 316 is constant during operation. As noted above, the C-arm 310, as well as the associated detector 316 and source 314, are rotatable such that they may be moved through a plurality of different orientations, and in some instances different poses, relative to the subject 350, or other object disposed between the source 314 and detector 316. Thus, the source 314 and detector 316 may be used to obtain a stream of sequential x-ray images of the subject 350 at a plurality of orientations relative to the subject 350 as the C-arm 310 is rotated by the operator 340 between an initial and final pose. As noted above, this may correspond to rotation between any desired poses including rotation over an entire rotational range of the C-arm 310 or a portion of the rotational range of the C-arm 310.

In some examples, a medical procedure planning apparatus as described herein may be part of the controller 320 of the external imaging device 100. Alternatively or additionally, the medical procedure planning apparatus may be part of a separate computer, such as a desktop computer, a portable computer, and/or a remote or local server. In some examples, the medical procedure planning apparatus may include at least one processor, such as the controller 320 with associated non-transitory computer readable memory. The memory may include computer executable instructions, that when executed by the at least one processor, cause the apparatus to perform any of the methods disclosed herein.

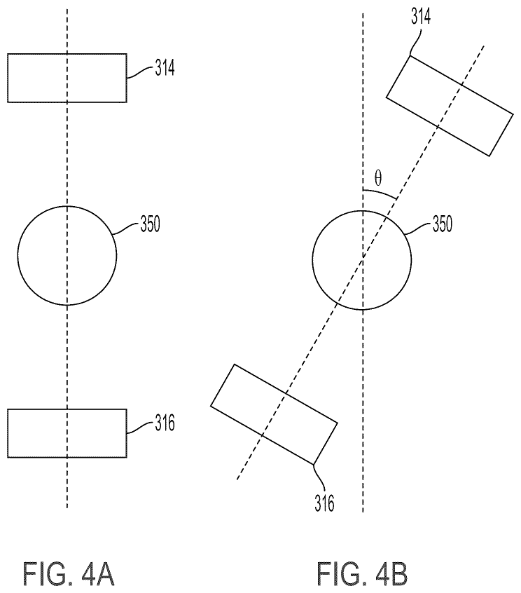

FIGS. 4A and 4B illustrate a schematic depiction of one example of different orientations of a source 314 and detector 316 of an external imaging device relative to a subject 350. As illustrated in the figure, and similar to the example described above relative to FIG. 3, the source 314 and detector 316 are disposed on opposing sides of the subject 350 with a predetermined constant pose between the source 314 and detector 316. In FIG. 4A, the source 314 and the detector 316 of the external imaging device are oriented in a vertical orientation, which may be referred to as a 0° orientation relative to the vertical axis of the system. In some examples, the vertical axis of the system may correspond to a local direction of gravity relative to the system. In FIG. 4B, the source 314 and the detector 316 have been rotated by an angle e relative to the vertical axis of the system such that the source 314 and the detector 316 have been rotated from the first orientation relative to the subject 350 to a second different orientation to view the subject 350 from a different perspective.

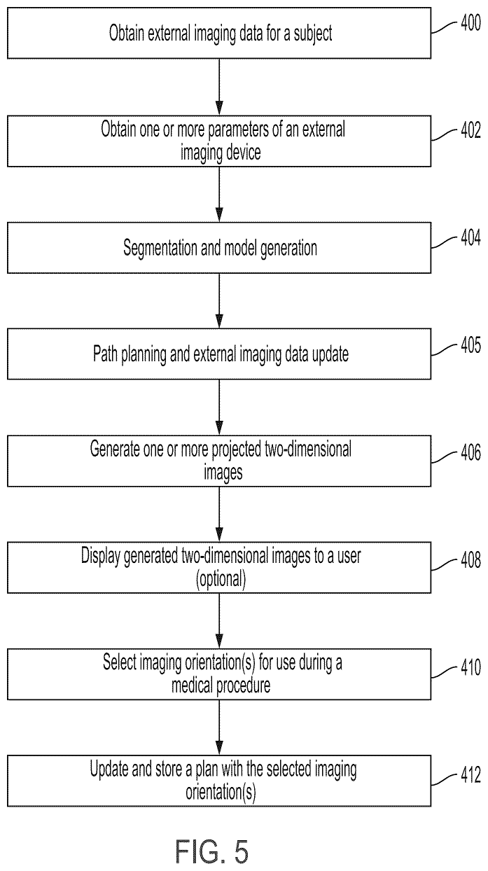

FIG. 5 depicts one example of a method for pre-planning imaging orientations for use with an external imaging device during a medical procedure to be performed on a subject. In the depicted method, at step 400 external imaging data (e.g., computed tomography (CT) data, MRI data, ultrasound data, etc.) related to a subject may be obtained. For example, external imaging data may correspond to preoperative CT scans used for diagnosis and/or planning purposes may be taken by a medical practitioner using a CT scanner (not depicted). Depending on the particular example, the resulting external imaging data may either be immediately processed using the methods disclosed herein and/or stored in non-transitory computer readable memory for future recall and usage. Depending on the procedure to be performed, this external imaging data may correspond to three-dimensional information of the anatomical structures and tissue within various portions of the subject's body including, but not limited to, a torso, limb, head, or other appropriate portion of the subject's body.

At 402, one or more parameters related to an external imaging device to be used during a medical procedure may be obtained. These parameter(s) may be previously stored in non-transitory computer readable memory for subsequent recall, input manually by a user, and/or automatically downloaded from an associated external imaging device as the disclosure is not limited in this fashion. In any case, the one or more parameters may correspond to parameters related to how the external imaging device captures an image of a subject. For example, appropriate parameters may include, but are not limited to, device geometry, source intensity, a relative spacing and/or orientation (e.g., pose) of a source and detector of the external imaging device, a range of motion of the external imaging device, a position of the x-ray source and the detector relative to a subject, camera distortion matrix, image noise level, and/or any other appropriate parameter related to the imaging device.

To facilitate determining a planned pose of a flexible elongated device relative to a target tissue during one or more stages of a medical procedure, it may be desirable to generate a model of the anatomy of a subject. Accordingly, in some examples, the obtained external imaging data may be subjected to segmentation and model generation at 404 The resulting model may correspond to a model of one or more portions of the anatomy of the subject. For example, the model may correspond to a model of the lungs of a subject undergoing a pulmonary procedure. Using the model and a location of a target tissue within the subject, a planned path for insertion and operation of the flexible elongated device may be determined for one or more portions of a medical procedure at 405. This may include determining a path through the one or more modeled portions of the subject's anatomy going from an entry location such as a natural or artificial orifice formed in the subject's body to a location adjacent to a target tissue within the subject's body. For example, the planned pose for the flexible elongated device may extend from a mouth through the esophagus to a location within the lungs of a subject adjacent to a target tissue. Depending on the specific example, the planned pose may be determined manually by a practitioner and/or may be generated automatically by an appropriate path planning software module as the disclosure is not so limited. In some examples, the external imaging (e.g., CT) data may be updated to include information related to this planned pose of the flexible elongated device. For example, the model and planned pose may be used to update the intensities of the voxels included in the external imaging data corresponding to the planned pose. For example, the intensities of voxels in the in which (e.g., CT data) corresponding to the planned pose of the flexible elongated device during the medical procedure may be increased. In some examples, this may include increasing the intensity of these voxels to a maximum intensity associated with the external imaging data, or other appropriately large intensity, to provide a high contrast relative to adjacent tissue as would be expected for a metallic shaft of a flexible elongated device inserted into the anatomy of a subject during x-ray imaging.

An example of segmentation, model generation, and path planning for a medical procedure using external imaging data is described in International Patent Application WO 2018/195221 which is incorporated herein by reference in its entirety. However, it should be understood that any appropriate method for determining a planned pose of the flexible elongated device within the anatomy of a subject may be used including, for example, manual path planning processes.

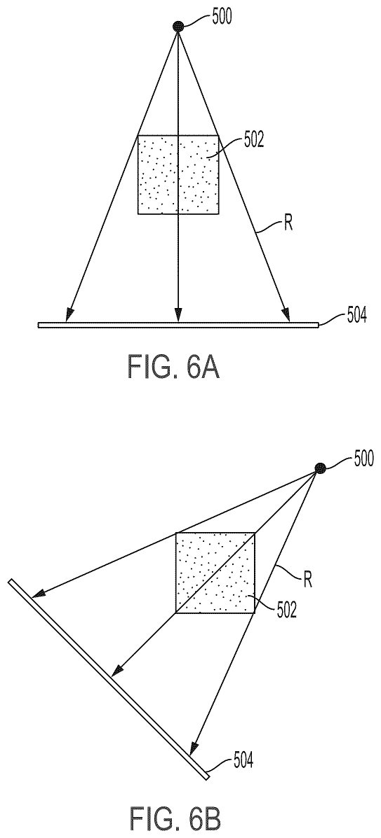

In some examples, once the external imaging (e.g., CT) data is updated with the planned pose of a flexible elongated device, one or more projected two-dimensional images, and in some instances a plurality of projected two-dimensional images may be generated at 406. This process is illustrated in FIGS. 6A-6B where a simulated source 500 is virtually positioned at a known location and orientation (i.e., pose) relative to a simulated receiver 504 of an external imaging device to be used. The relative poses of the source 500 and the receiver 504 may be determined based on the parameters of the external imaging device. The three dimensional voxels of the external imaging (e.g., CT) data 502 may be virtually positioned at an expected location and pose relative to the virtual source 500 and detector 504. As elaborated on below, the pose of the virtual source 500 and detector 504 relative to the external imaging data 502 may be used to generate a projected two dimensional image corresponding to the plane in which the virtual detector 504 is located in as elaborated on below. This process is illustrated in a first pose in FIG. 6A and may be repeated for any number of different orientations or poses as illustrated by the second orientation of the virtual source 500 and virtual detector 504 relative to the external imaging data 502 shown in FIG. 6B. For example, a plurality of projected two-dimensional images may be formed at different orientations distributed either uniformly or non-uniformly across a range of motion of an external imaging device.

In some examples, the projected two-dimensional images depicted in FIGS. 6A-6B may be formed using a method such as digital reconstruction of radiograph images. Such an algorithm may generate the one or more projected two-dimensional images using the one or more external imaging device parameters noted above and the relative pose of the virtual source 500 and detector 504 relative to the external imaging data 502. When generating a projected two dimensional image, the algorithm may calculate a straight line (ray) from the source 500 to each pixel on the detector 504 based on the 3D coordinates, see the arrows R in FIGS. 6A and 6B. The algorithm may then trace each point (e.g., voxel) along each ray to see if it intersects with the three-dimensional volume of the external imaging data 502. If a ray intersects with one or more voxels of the external imaging data, it will fetch the intensity value (e.g., Hounsfield unit (HU) value) for that particular voxel and convert the intensity value to an attenuation coefficient. The algorithm may sum the determined attenuation coefficients along each ray associated with the separate pixels of the virtual detector and the resulting summed attenuation coefficient may be used as the intensity value for the pixel of the detector associated with that particular ray. By doing this calculation for each pixel of the virtual detector 504, a final projected two-dimensional image may be generated. As noted previously, this process may be done for any number of different orientations of the virtual source and detector relative to the three-dimensional volume of the external imaging data 502 to provide a plurality of projected two-dimensional images associated with the different orientations of an external imaging device. For example, projected two-dimensional images may be generated for different orientations across either a portion and/or a full range of motion of an external imaging device. In one such example, the external imaging device may be a C-arm fluoroscope and the plurality of different orientations may correspond to a plurality of different C-arm angles which may either be uniformly distributed, or non-uniformly distributed, across a range of motion of the C-arm. Of course, it should be understood that different types of external imaging devices may also be used.

It should be understood that while a particular method for generating the projected two-dimensional images using the external imaging data has been described above, any appropriate method for generating projected two-dimensional images may be used.

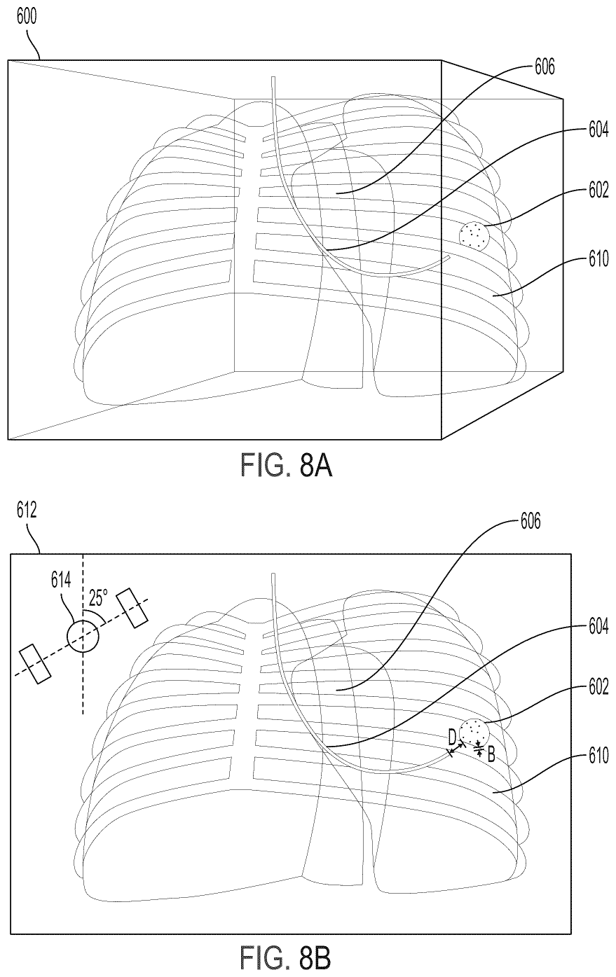

In some examples, it may be desirable to display the plurality of two-dimensional images to a user, see step 408 in FIG. 5. For example, the images may optionally be displayed sequentially to a user where the projected two dimensional images may sequentially progress from a first pose and/or orientation to a final pose and/or orientation along a range of motion of the external imaging device. In one such example, the sequential projected two-dimensional images may correspond to C-arm angles ranging from 0° to a maximum C-arm angle of the C-arm fluoroscope. Regardless, the plurality of projected two-dimensional images may be displayed to the user in any appropriate fashion including, but not limited to, a video sequence, manually scrolling through the sequential image stack, and/or any other appropriate type of presentation. Additionally, as described further below in regards to FIGS. 8A and 8B, the images may include various types of information for viewing by a user including, but not limited to, a planned pose of a flexible elongated device, a location of a target tissue (e.g., a lesion, tumor, or other desired target tissue) within the projected images, and/or any other appropriate information. In some examples, to further emphasize the location of the target tissue with the images, the software can overlay the target tissue on the projected two dimensional images.

At 410 one or more orientations may be selected for use during a medical procedure. The one or more orientations may be selected in any appropriate fashion. For example, in one example, a user may manually select the one or more imaging orientations by manually selecting one or more of the plurality of projected two dimensional images. Alternatively, the one or more orientations may be selected based on one or more appropriate criteria evaluated by an associated selection algorithm. Examples of appropriate methods for selecting one or more imaging orientations are provided below.

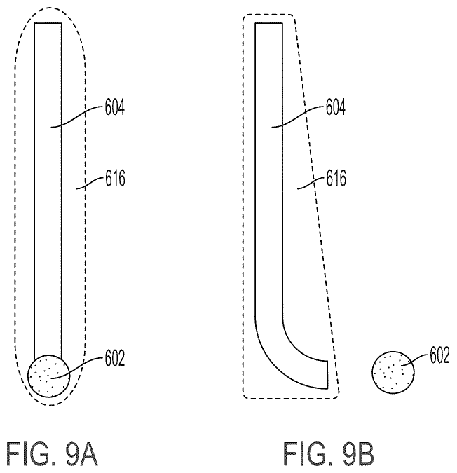

In one example, one or more orientations may be selected based at least in part on a curvature of the simulated flexible elongated device 604 within the projected two dimensional image (e.g., the curvature of a catheter or endoscope along a planned path). In one such example, a minimum bounding area 616 of the projected two dimensional curve of the flexible elongated device 604 in the projected plane may be used to determine a desired imaging orientation. For example, as shown in FIG. 9A, and 9B, the minimum bounding area 616 enclosing the flexible elongated device 604 may be determined for each projected two dimensional image. This is illustrated by the smaller bounding area 616 in FIG. 9A where the flexible elongated device 604 and target tissue 602 are either overlapping within a projected two dimensional image as compared to the larger minimum bounding area 616 in FIG. 9B where the profile of the flexible elongated device 604 is spaced apart from and oriented towards the target tissue 602. Thus, larger minimum bounding areas may be associated with images where the relative pose of the target tissue 602 and flexible elongated device 604 may be more readily viewed. In another example, an accumulated curvature of the flexible elongated device 604 may be determined for each projected two dimensional image by summing the curvature along the projected planned pose of the flexible elongated device in each image. As shown in FIGS. 9A and 9B, the summed curvature along a length of the flexible elongated device 604 within the projected two dimensional images is larger in FIG. 9B where the relative pose of the target tissue 602 and flexible elongated device 604 may be more readily viewed. Thus, after determining the minimum bounding area and/or accumulated curvature, the projected two dimensional image with a maximum value of the minimum bounding area and/or accumulated curvature for the projected planned pose of the flexible elongated device in the image may then be selected for a proposed imaging orientation during the procedure.

In another example, a geometrical relationship between the projected flexible elongated device and the target tissue in the projected two dimensional image may be used to select an orientation for use during a medical procedure. For example, in some examples, a distance D corresponding to a separation distance between a distal end portion of the flexible elongated device 604 in a planned pose and the target tissue 602 within the projected two dimensional image may be determined for each image of the plurality of projected images, for example distance D in FIG. 8B. The projected two dimensional image with the smallest separation distance between the distal end portion of the flexible elongated device 604 in the planned pose and the target tissue 602 may then be selected.

In yet another example, a visibility of a target tissue within the plurality of two-dimensional projected images may be used to determine one or more imaging orientations for use during a medical procedure. In some such examples, a target region may correspond to a tissue region that includes the target tissue. For example, the target region may be approximately 2 to 3 times a size of, and be collocated with, the target tissue. The target region may include both the target tissue and tissue surrounding the target tissue. The target region may be appropriately registered with the target tissue within the external imaging data and may also be included in the subsequently generated projected two-dimensional images. Various metrics related to improving visibility of the target tissue relative to other anatomical structures may then be used to select one or more orientations for use during a medical procedure. For example, appropriate metrics may include, but are not limited to, signal to noise ratio (e.g., contrast ratio between target tissue and surrounding tissue in the target region), a ratio of average value of pixels to a standard deviation of pixel values in the target region, total variation of image gradient in the target region, structure similarity (SSIM), and/or any other appropriate metric for selecting one or more imaging orientations based on an analysis of the plurality of projected two-dimensional images.

In another example, a geometrical relationship between the projected target tissue relative to one or more projected bony structures or other highly visible structures within the plurality of projected two-dimensional images may be used to select one or more imaging orientations for use during a medical procedure. For example, a separation distance B corresponding to a minimum distance between the target tissue and the one or more bony structures within the plurality of projected two-dimensional images may be determined for each image, see FIG. 8B. Threshold intensities may be used, at least in part, to help facilitate identification of the bony structures or other highly visible structures in the plurality of projected two dimensional images. For example, pixel intensities in the projected two dimensional image(s) may be compared to a threshold intensity, and pixels with intensities greater than the threshold intensity may be identified as a bony structure, or other similar anatomical structure that might interfere with visualization of the target tissue. However, other appropriate ways to determine the minimum distance may be used as well. The minimum separation distance between the target tissue and the one or more identified bony structures may then be identified for each image of the plurality of projected two dimensional images. One or more orientations may then be selected based on this determined spacing of the target tissue from the one or more bony structures within the projected two-dimensional images. For example, an orientation corresponding to a projected two dimensional image having a largest minimum spacing may correspond to an orientation in which the target tissue is most unobstructed by the surrounding bony structures.

While several methods for automatically determining one or more imaging orientations for use with an external imaging device during a medical procedure are provided above, it should be understood that these methods may either be used in combination with one another, separately, and/or other appropriate methods for determining the one or more imaging orientations may be used. Additionally, in some examples, a system may automatically determine proposed imaging orientations using the above described methods without user intervention. However, in other examples, the proposed orientations for use during a medical procedure may be recommended by the software to a user for review and approval. This may include, for example, displaying the orientations and corresponding projected two-dimensional images to a user on a display for approval. The recommended imaging orientations of the external imaging device may be displayed to the medical practitioner in any appropriate fashion including, but not limited to, numerical outputs, visual outputs such as orientation icons, combinations of the foregoing, and/or any other appropriate type of indication. See, for example, the orientation icon 612 shown in FIG. 8B. In some examples, an indication regarding one or more medical activities associated with the one or more proposed imaging orientations may also be displayed to the medical practitioner. This may include the display of appropriate icons and/or text to indicate the medical activity. For example, a first set of proposed imaging orientations may be proposed for a first medical activity and a second set of proposed imaging orientations may be proposed for a second medical activity where the optimal viewing angles may be different. After the proposed imaging orientations have been presented to the user, the user may then manually choose one or more of the proposed orientations for inclusion in a medical plan.

After the one or more orientations are selected, the one or more selected imaging orientations may then be used to update a plan at 412. For example, the selected one or more imaging orientations for use with the external imaging device may be stored with the updated plan on non-transitory computer readable memory for future recall for reference purposes and/or use during a medical procedure. In some examples, the plan may be uploaded, or otherwise transferred to, a system including an external imaging device. A medical practitioner may then use the plan during a medical procedure where the one or more selected imaging orientations may be displayed as recommendations to the medical practitioner to provide guidance regarding which orientations of the external imaging device should be viewed during the medical procedure. The recommended orientations may be displayed to the medical practitioner in any appropriate fashion including, but not limited to, numerical outputs, visual outputs such as orientation icons, combinations of the foregoing, and/or any other appropriate type of indication.

### Example: Determining Imaging Orientations

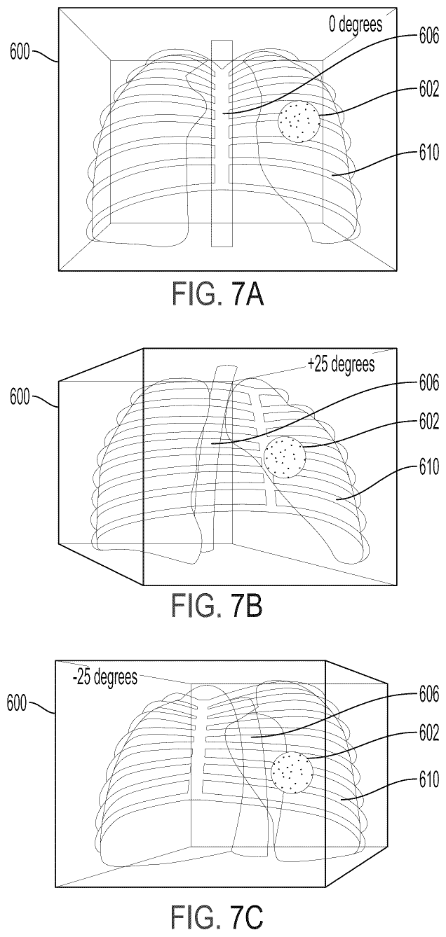

FIGS. 7A-7C show an example of a three dimensional volume 600 including data from a Computed Tomography (CT) scan of a subject's torso. The scans include information such as the location of a target tissue (e.g., a tumor, lesion, or other appropriate structure) as well as bony structures such as the spine 606 and ribs 610 of the subject. FIG. 7A is illustrated from a forward perspective with a viewing angle of 0 degrees. FIGS. 7B and 7C show the same CT scan at +25 degrees and −25 degrees respectively which changes the relative positioning of the different structures included in the field of view of the presented perspectives. These different perspectives may thus be used to determine what a two dimensional x-ray image of a subject might look like prior to an operation being performed.

FIGS. 8A and 8B depict the use of a three dimensional volume 600 including information from the CT scan of FIGS. 7A-7C to generate a projected two dimensional image 612 for determining one or more imaging orientations for a medical procedure. The projected two dimensional image may be generated using any of the methods described above. In the depicted example, the CT data includes a planned pose of a flexible elongated device 604. The planned pose may be depicted by voxels within the three dimensional volume 600 and pixels within the projected two dimensional image 612 that have an elevated intensity relative to the surrounding background voxels and/or pixels. This may permit the flexible elongated device's 604 pose within the three dimensional volume, and subsequently generated projected two dimensional image 612, to be easily identified by an associated algorithm and/or user. The CT data may also include an identified location of target tissue 602. In some examples, the location of the target tissue may be included in the CT data, and as depicted in FIG. 8B, may be overlaid on the projected two dimensional image 612 to enhance visualization of the target tissue relative to other features in the image. In some examples, and as described above, the projected two dimensional image 612 may also include an indication of an orientation of an external medical imaging device that would correspond to the presented image. This may include, for example, an orientation icon 614 shown in FIG. 8B, text, or any other appropriate indicator of the imaging orientation associated with the image.

In addition to the above, FIG. 8B illustrates the separation distance D between a distal end portion of the flexible elongated device 604 and the target tissue 602 within the projected two dimensional image 612. The figure also illustrates a minimum distance B between the target tissue 602 and the one or more bony structures such as the adjacent rib 610 in the projected two dimensional image 612. As detailed above, information such as this may be used either by a user and/or an algorithm to help select appropriate orientations for use during a medical procedure.

While the above examples are generally described as generating projected two dimensional images from CT data, other external imaging systems configured to provide different types of external imaging data may also be used as discussed previously. This may include, for example, magnetic resonance imaging (MRI), fluoroscopy, thermography, ultrasound, optical coherence tomography (OCT), thermal imaging, impedance imaging, laser imaging, nanotube x-ray imaging, and/or any other appropriate type of imaging system. Accordingly, it should be understood that any appropriate type of external imaging data can be used to generate the desired projected two dimensional images.

It should be understood that the above-described and other methods disclosed herein, may be implemented in any appropriate manner. For example, in some examples, the methods disclosed herein may be embodied as processor executable instructions stored in non-transitory computer readable memory that when executed cause a system to perform any of the methods disclosed herein. Additionally, it should be understood that while a single processor is described, examples in which the methods disclosed herein are implemented by any number of processors working either in combination and/or separately from one another are contemplated.

The above method may be implemented by one or more controllers including at least one processor as disclosed herein. The method may be embodied as computer executable instructions stored on non-transitory computer readable memory associated with the at least one processor such that when executed by the at least one processor the system may perform any of the actions related to the methods disclosed herein. Additionally, it should be understood that the disclosed order of the steps is illustrative and that the disclosed steps may be performed in a different order, simultaneously, and/or may include one or more additional intermediate steps not shown as the disclosure is not so limited.

The above-described examples of the technology described herein can be implemented in any of numerous ways. For example, the examples may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device including one or more processors may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone, tablet, or any other suitable portable or fixed electronic device.

Also, a computing device may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools.

In this respect, the examples described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various examples discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium”, “computer-readable memory”, or other similar term encompasses only a non-transitory computer-readable medium that can be considered to be a manufacture (e.g., article of manufacture) or a fluoroscope. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this example, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various examples.

The examples described herein may be embodied as a method, of which an example has been provided. The acts performed as part of the method may be ordered in any suitable way. Accordingly, examples may be constructed in which acts are performed in an order different than illustrated, which may include performing some acts simultaneously, even though shown as sequential acts in illustrative examples.

Further, some actions are described as taken by a “user.” It should be appreciated that a “user” need not be a single individual, and that in some examples, actions attributable to a “user” may be performed by a team of individuals and/or an individual in combination with computer-assisted tools or other mechanisms.

While the present teachings have been described in conjunction with various examples and examples, it is not intended that the present teachings be limited to such examples or examples. On the contrary, the present teachings encompass various alternatives, modifications, and equivalents, as will be appreciated by those of skill in the art. Accordingly, the foregoing description and drawings are by way of example only.

1. A method comprising:
obtaining computed tomography (CT) data for a subject, wherein the CT data includes a target tissue of the subject;
obtaining a planned pose of a flexible elongated device within an anatomical structure of the subject; and
based on the CT data, the planned pose of the flexible elongated device, and one or more parameters of an external imaging device, forming one or more projected two dimensional images.

2. The method of claim 1, wherein the external imaging device comprises an x-ray imaging device.

3. The method of claim 1, wherein forming the one or more projected two dimensional images includes forming a plurality of projected two dimensional images at a plurality of imaging orientations of the external imaging device.

4. The method of claim 1, further comprising determining one or more imaging orientations of the external imaging device for use during a medical procedure based at least in part on the one or more projected two dimensional images.

5. The method of claim 4, wherein determining the one or more imaging orientations of the external imaging device includes receiving a manual selection of one or more of the one or more projected two dimensional images.

6. The method of claim 4, wherein determining the one or more imaging orientations of the external imaging device includes determining a projected two dimensional image of the one or more projected two dimensional images with a maximum bounding area of the flexible elongated device.

7. The method of claim 4, wherein determining the one or more imaging orientations of the external imaging device includes determining a projected two dimensional image of the one or more projected two dimensional images with a maximum accumulated curvature value along the planned pose of the flexible elongated device.

8. The method of claim 4, wherein determining the one or more of the imaging orientations of the external imaging device includes determining a separation distance between the target tissue and a distal end portion of the flexible elongated device in the one or more projected two dimensional images.

9. The method of claim 4, wherein determining the one or more imaging orientations of the external imaging device includes determining a separation distance between the target tissue and one or more bony structures in the one or more projected two dimensional images.

10. The method of claim 9, further comprising determining a position of the one or more bony structures in the one or more projected two dimensional images based at least in part on a threshold intensity.

11. The method of claim 4, wherein determining the one or more imaging orientations of the external imaging device includes determining a visibility of the target tissue in the one or more projected two dimensional images.

12. The method of claim 4, further comprising displaying an indication to a user regarding one or more medical activities associated with the determined one or more imaging orientations.

13. The method of claim 4, further comprising storing the determined one or more imaging orientations on non-transitory computer readable memory.

14. The method of claim 4, wherein the one or more imaging orientations comprise one or more imaging orientations of a C-arm fluoroscope.

15. The method of claim 4, further comprising outputting an indication of the one or more imaging orientations to a user.

16. The method of claim 1, wherein the planned pose is based on a planned path of the flexible elongated device into the anatomical structure of the subject.

17. The method of claim 1, further comprising increasing an intensity of voxels in the CT data corresponding to the planned pose of the flexible elongated device.

18. The method of claim 1, wherein the anatomical structure includes lungs of the subject.

19. A non-transitory computer readable memory including instructions that when executed by one or more processors performs a method comprising:
obtaining computed tomography (CT) data for a subject, wherein the CT data includes a target tissue of the subject;
obtaining a planned pose of a flexible elongated device within an anatomical structure of the subject; and
based on the CT data, the planned pose of the flexible elongated device, and one or more parameters of an external imaging device, forming one or more projected two dimensional images.

20-37. (canceled)

38. The non-transitory computer readable memory of claim 19, wherein the method further comprises determining one or more imaging orientations of the external imaging device for use during a medical procedure based at least in part on the one or more projected two dimensional images.
